(19) United States
(12) Patent Application Publication
(10) Pub. No.: US 2019/0357834 A1
(43) Pub. Date: Nov. 28, 2019

(54) DRIVER AND PASSENGER HEALTH AND SLEEP INTERACTION

(71) Applicant: KONINKLIJKE PHILIPS N.V.

(72) Inventors: Ronaldus Maria AARTS, Geldrop (NL); Adrienne HEINRICH, Boxtel (NL)

(21) Appl. No.: 16/484,840

(22) PCT Filed: Feb. 9, 2018
(86) PCT No.: PCT/EP2018/053316

§ 371 Date: Aug. 9, 2019

Related U.S. Application Data
(60) Provisional application No. 62/457,433, filed on Feb. 10, 2017.
(60) Provisional application No. 62/598,711, filed on Dec. 14, 2017.

(51) Int. Cl. A61B 5/18; G08B 21/06; A61B 5/0205; A61B 5/00; B60Q 9/00

(52) U.S. Cl. CPC A61B 5/18; G08B 21/06; A61B 5/02405; A61B 5/7246; B60Q 9/00; A61B 5/0205

(57) ABSTRACT

In one embodiment, an apparatus is presented that receives a driving style of a driver, senses a parameter or parameters of a driver and/or at least one passenger within a vehicle, correlates the parameter(s) to the driving style, and triggers feedback to the driver of the correlated parameter(s) of the at least one passenger or to the passenger of the correlated parameters parameter(s) of the driver. The invention provides, among other features, a mechanism to increase positive interactions between the driver and the passenger(s) and to decrease or avoid negative interactions, which leads to a safer use of the vehicle based on the correlations between measured health/well-being data and driving style or behavior.

### RELATED APPLICATIONS

This application claims the benefit of and priority to U.S. Provisional Application Ser. No. 62/457,433, filed Feb. 10, 2017, which is incorporated herein by reference in its entirety.

### FIELD OF THE INVENTION

The present invention is generally related to vehicle safety, and in particular, managing vehicle occupant interactions within a vehicle or among multiple vehicles to promote safety.

### BACKGROUND OF THE INVENTION

Generally, drivers and passengers have difficulty perceiving what the other is experiencing or their health status. Due to the positioning of car seats, it can be uncomfortable to look at the other person for extended periods of time or such views may be at least partially obstructed. Further, during a long trip, a driver or passenger's attention may be focused elsewhere. Further, passengers may or may not interact positively with each other or the driver of a vehicle. In cases where a passenger's behavior negatively impacts the driver, accidents may happen or frustrate the driver, other passengers, or even other participants (e.g., other cars in the vicinity).

International patent application publication No. WO2014121182A1 (hereinafter, “the '182 Pub”, with supporting disclosure from this publication in parenthesis) is described in the context of managing operator stress in the vehicle, such as to prevent road rage (see, e.g., the background in the '182 Pub), and describes (e.g., beginning at page 3, line 30) at least a portion of a group of sensors that can collect or can be configured to collect information (e.g., data, metadata, and/or signaling) indicative of operational features of a vehicle. For example, at least one sensor (e.g., one sensor, two sensors, more than two sensors, or the like) of the group of sensors can detect or can be configured to detect motion of the vehicle. The '182 Pub further describes (beginning at page 4, line 8) that at least another portion of the group of sensors can collect or can be configured to collect information indicative of behavior of an occupant of the vehicle, such as the operator of the vehicle or a passenger of the vehicle. The '182 Pub further describes (beginning at page 2, line 12) that three types of information can be combined or otherwise integrated to generate a rich group of data, metadata, and/or signaling that can be utilized or otherwise leveraged to generate a condition metric representative of the emotional state of the vehicle operator, and that in one scenario, the condition metric can be supplied by rendering it to the operator of the vehicle.

Though mitigating the potential for conflict on the roadways among passengers of different vehicles is beneficial to a community of drivers, sometimes stress among passengers within a vehicle may lead to safety concerns on the road.

### SUMMARY OF THE INVENTION

One object of the present invention is to develop a vehicle occupant interaction system that manages the effect of a behavior and/or condition of one vehicle occupant on another occupant of the vehicle. To better address such concerns, in a first aspect of the invention, an apparatus is presented that receives a driving style of a driver, senses a parameter or parameters of a driver and/or at least one passenger within a vehicle, correlates the parameter(s) to the driving style, and triggers feedback to the driver of the correlated parameter(s) of the at least one passenger or to the passenger of the correlated parameters parameter(s) of the driver. The invention provides, among other features, a mechanism to increase positive interactions between the driver and the passenger(s) and/or to decrease or avoid negative interactions, which leads to a safer use of the vehicle based on the correlations between measured health/well-being data and driving style or behavior.

In one embodiment, the parameters correspond to one or any combination of heart rate, heart rate variability, electrodermal activity, accelerometer data, indicators of stress, indicators of anxiety, or indicators of motion sickness. For instance, the apparatus measures (or receives measures) pertaining to a change in health or well-being (e.g., stress, anxiety, motion sickness, etc.) of say, the passenger, which is correlated to the driving style as indicated by the vehicle movement information (e.g., fast accelerations, speed, odd movements, etc.). Similar measures may be received from the driver, which may be the result of the passenger behavior (e.g., upset, worried, etc.) that results from the driver's style of driving. By monitoring these parameters, occupants in a vehicle are informed of real time information to enable a suitable reaction to positively impact the driving experience.

In one embodiment, the apparatus triggers the feedback by communicating a signal to one or any combination of a tactile device, visual device, audio device, or audio-visual device, wherein the feedback comprises one or any combination of tactile feedback, visual feedback, or audio feedback. For instance, in the case of feedback to the driver, the feedback may be presented, in a haptic manner by a tactile device embedded within the steering wheel of the vehicle, armrest, seat, gear shift, etc.), or embedded within a wearable device worn by the driver, vibratory alerts presented on a wearable or mobile device possessed by the driver or in structures of the vehicle. In addition to, or in lieu of tactile feedback, the feedback to the driver may be presented visually using a vehicle display screen or dashboard (or via user interface functionality of the wearable or mobile device) with text or warning lights, or via eyewear (e.g., Google glass), and/or audibly (e.g., using a headset, vehicle speaker, or beep or buzzard of the driver's wearable device and/or mobile device). Similar mechanisms of feedback may be presented to the passenger (e.g., using his or her own wearable, mobile device, and/or structures within the vehicle, such as a nearby speaker, motors/actuators in an armrest, seat, etc.). The feedback influences each occupant to change their respective behavior to make for a positive driving experience, and safe travels.

In one embodiment, the apparatus may be configured to communicate the signal without alerting the passenger of the feedback to the driver or without alerting the driver to the feedback to the at least one passenger (e.g., via haptic feedback, textual feedback, and/or the like). In other words, the feedback may be presented inconspicuously to the intended recipient. Such feedback may prevent an embarrassing or awkward situation and/or reduce the chance of further escalation of conflict, facilitating more harmony in travel through the avoidance of conflict.

In one embodiment, an apparatus is further configured to receive one or more parameters sensed from one or more occupants in one or more other vehicles, the one or more parameters comprising physiological information, emotional information, or a combination of physiological and emotional information, wherein the correlation and the trigger is based on the one or more occupants. In doing so, the apparatus triggers feedback on how his or her driving behavior is negatively impacting others driving around them, helping to reduce conflict.

In one embodiment, an apparatus is configured to receive one or more first parameters sensed from a driver of a vehicle and one or more second parameters sensed from a passenger in the vehicle, the one or more first parameters and the one or more second parameters each comprising physiological information, behavioral information, or a combination of physiological and behavioral information; predict respective sleepiness levels of the driver and the passenger based on the received one or more first and second parameters; and trigger feedback to either the passenger in the vehicle based on the predicted sleepiness level for the driver or to the driver based on the predicted sleepiness level for the passenger. For instance, the sleep state of the driver and one or more passengers is monitored. In the case where both the driver and passenger wish to remain awake, and the driver's attention is decreasing, feedback is sent to the passenger to influence the passenger to direct his or her attention to keeping the driver awake, including by taking such action(s) as talking to the driver, turning on the radio, changing vehicle environmental settings (e.g., colder air), etc. If the driver expects to be kept awake by the passenger and the passenger's attention is sensed as being reduced, the driver receives feedback and may request that the passenger remain alert. Through the mutual monitoring of the sleepiness levels of occupants within the vehicle, the safety of each occupant is ensured through collaborative effort that is computer-assisted.

In one embodiment, an apparatus is configured to receive a drive plan including a route and driving time for a vehicle comprising a driver and a passenger; determine a time for the passenger to commence a nap or inattentive period lasting a defined duration based on the received drive plan; and trigger a recommendation to the passenger about the time. For instance, the planned route and driving times are taken into account when scheduling the best time for the passenger to take a nap (e.g., to be fresh and alert when, say, the passenger switches roles with the driver) or be inattentive.

In one embodiment, an apparatus is further configured to determine the time based on one or any combination of information about a sleep behavior of the driver, information about a sleep behavior of the passenger, information about the safety of travel along the route, information about complexity of travel along the route, elapsed driving time by the driver, time of day, traffic, construction, or weather. For instance, the apparatus recommends naps (or allows for the passenger to be inattentive in some embodiments) on safer route stretches (e.g., with lower accident occurrences and/or presenting less challenge to driving skills) and/or according to other factors including elapsed driving time of the driver, time of day (e.g., people tend to feel sleepier earlier in the night), etc. The apparatus enables an intelligent decision on a recommended nap or inattentive commencement time that enables safe travel.

In one embodiment, at least one of the information is received from a source external to the vehicle. For instance, the apparatus may use information stored in an external data base that stores user data, including personal information (e.g., sleep patterns of the driver and/or passenger, statistics on road accidents, traffic patterns, etc.), where the external database alleviates the need for memory capacity for a device or devices within the vehicle, particularly battery-powered devices.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that illustrates an example vehicle in which a vehicle occupant interaction system is used, in accordance with an embodiment of the invention.

FIG. 2 is a schematic diagram that illustrates an example wearable device in which all or a portion of the functionality of a vehicle occupant interaction system may be implemented, in accordance with an embodiment of the invention.

FIG. 3 is a schematic diagram that illustrates an example mobile device in which all or a portion of the functionality of a vehicle occupant interaction system may be implemented, in accordance with an embodiment of the invention.

FIG. 4 is a schematic diagram that illustrates an example vehicle processing unit in which in which all or a portion of the functionality of a vehicle occupant interaction system may be implemented, in accordance with an embodiment of the invention.

FIG. 5 is a flow diagram that illustrates an example vehicle occupant interaction method, in accordance with an embodiment of the invention.

FIG. 6 is a flow diagram that illustrates another example vehicle occupant interaction method, in accordance with an embodiment of the invention.

FIG. 7 is a flow diagram that illustrates another example vehicle occupant interaction method, in accordance with an embodiment of the invention.

### DETAILED DESCRIPTION OF EMBODIMENTS

Disclosed herein are certain embodiments of a vehicle occupant interaction system that may improve the safety of vehicular travel by increasing the positive interactions between a driver of the vehicle (i.e., the human occupant of the vehicle that controls the navigation of the vehicle) and one or more passengers within the vehicle, and/or by decreasing or avoiding negative interactions, leading to a potentially safer use of the vehicle. In one embodiment of a vehicle occupant interaction system, an apparatus comprises memory and one or more processors that monitor the health and/or well-being of the driver and/or passenger and the driving style of the driver. Such monitoring may be performed by one or more sensors embedded within (or attached externally) to structures of the vehicle, in wearable(s) attached to the occupants, in mobile devices of the occupants, or any combination thereof. The apparatus correlates the driving style to the health parameter(s), and triggers feedback to one occupant about changes in the health or well-being of the other occupant to facilitate a positive and safe driving experience for all occupants. In some embodiments, the apparatus may use the monitored health parameters to predict a level of sleepiness of the occupants. In some embodiments, the apparatus may use information about a drive plan to recommend a nap/inattentive time for a passenger during a given trip. The recommendation seeks nap/inattentive times during travel routes that pose a lower challenge to driving and/or are safe to navigate without passenger attentiveness.

Digressing briefly, negative interactions among occupants of a vehicle may present a negative driving experience, and possibly lead to unaddressed frustrations and even accidents. By providing computer-assisted intelligence about vehicle occupant health and well-being, certain embodiments of a vehicle occupant interaction system can mitigate the risk of having such negative experiences and provide for positive and safe travel for all occupants involved.

Having summarized certain features of a vehicle occupant interaction system of the present disclosure, reference will now be made in detail to the description of a vehicle occupant interaction system as illustrated in the drawings. While a vehicle occupant interaction system will be described in connection with these drawings, there is no intent to limit the vehicle occupant interaction system to the embodiment or embodiments disclosed herein. For instance, though described primarily in the context managing the interactions among occupants in one vehicle, in some embodiments, interactions among occupants of multiple vehicles may be managed according to the vehicle occupant interaction system. Further, although the description identifies or describes specifics of one or more embodiments, such specifics are not necessarily part of every embodiment, nor are all various stated advantages necessarily associated with a single embodiment or all embodiments. On the contrary, the intent is to cover all alternatives, modifications and equivalents consistent with the disclosure as defined by the appended claims. Further, it should be appreciated in the context of the present disclosure that the claims are not necessarily limited to the particular embodiments set out in the description.

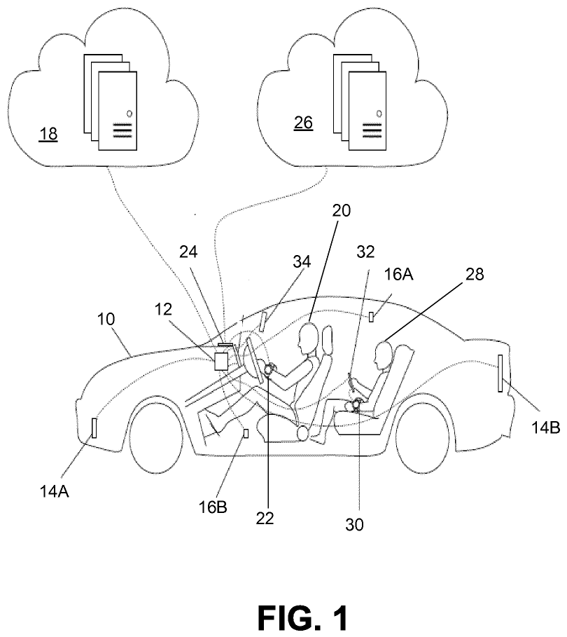

Referring now to FIG. 1, shown is an example vehicle 10 in which certain embodiments of a vehicle occupant interaction system may be implemented. It should be appreciated by one having ordinary skill in the art in the context of the present disclosure that the vehicle 10 is one example among many, and that some embodiments of a vehicle occupant interaction system may be used in other types of vehicles than the type depicted in FIG. 1. FIG. 1 illustrates the vehicle 10 having a vehicle processing unit 12, external vehicle sensors 14 (e.g., front 14A and rear 14B sensors), and internal vehicle sensors 16 (e.g., 16A and 16B). Note that the quantity of sensors 14, 16 and vehicle processing unit 12 is illustrative of one embodiment, and that in some embodiments, fewer or greater quantities of one or more of these types of components may be used. The internal vehicle sensors 16 are located in the cabin of the vehicle 10. The external vehicle sensors 14 are located on the exterior of the vehicle 10. The external vehicle sensors 14 and internal vehicle sensors 16 are capable of communicating with the vehicle processing unit 12, such as via a wireless medium (e.g., Bluetooth, near field communications (NFC), and/or one of various known light-coding technologies, among others) and/or wired medium (e.g., over a CAN bus or busses). The internal vehicle sensors 16 may include at least one of temperature sensors, microphones, cameras, light sensors, pressure sensors, accelerometers, proximity sensors, including beacons, radio frequency identification (RFID) or other coded light technologies, among other sensors. The external vehicle sensors 14 may include at least one of temperature sensors, sensors to measures precipitation and/or humidity, microphones, cameras, light sensors, pressure sensors, accelerometers, etc. In some embodiments, the vehicle 10 includes a geographic location sensor (e.g., a Global Navigation Satellite Systems (GNSS) receiver, including Global Position Systems (GPS) receiver, among others). The geographic location sensor provides location coordinates (e.g., latitude, longitude, altitude).

FIG. 1 further illustrates the vehicle processing unit 12 capable of communicating with at least one cloud (e.g., cloud 1) 18. That is, the vehicle processing unit 12 is capable of communicating (e.g., via telemetry, such as according to one or more networks configured according to, say, the Global System for Mobile Communications or GSM standard, among others) with one or more devices of the cloud platform (the cloud 18). The vehicle 10 also includes vehicle sensors related to the operation of the vehicle 10 (e.g., speed, braking, turning of the steering wheel, turning of the wheels, etc.). The vehicle 10 is capable of being driven by a (human) driver 20 that primarily controls navigation (e.g., direction, vehicle speed, acceleration, etc.) of the vehicle 10.

The driver 20 may drive the vehicle 10 while wearing a wearable 22 (herein, also referred to as the driver wearable or wearable device). The driver wearable 22 may include, for example, a Philips Health Watch or another fitness tracker or smartwatch. In some embodiments, the driver wearable 22 may include a chest strap, arm band, ear piece, necklace, belt, clothing, headband, or another type of wearable form factor. In some embodiments, the driver wearable 22 may be an implantable device, which may include biocompatible sensors that reside underneath the skin or are implanted elsewhere. The driver 20 may also wear the driver wearable 22 when he is not driving the vehicle 10. The driver 20 may further drive the vehicle 10 while in possession of his driver mobile device 24 (e.g., smart phone, tablet, laptop, notebook, computer, etc.) present in the vehicle 10. The driver wearable 22 is capable of communicating (e.g., via Bluetooth, 802.11, NFC, etc.) with the driver mobile device 24 and mobile software applications (“apps”) residing thereon and/or the vehicle processing unit 12. The driver mobile device 24 is capable of communicating with at least one cloud (e.g., cloud 2) 26. In some cases, the driver mobile device 24 is capable of communicating with the vehicle processing unit 12. At times, a passenger 28 may ride in the vehicle 10 with the driver 20. In some cases, the passenger 28 may wear a wearable 30 (also referred to herein as a passenger wearable or wearable device). In some cases, a passenger mobile device 32 (e.g., smart phone, tablet, laptop, notebook, computer, etc.) may be present with the passenger 28 in the vehicle 10. The passenger wearable 30 is capable of communicating with the passenger mobile device 32. The passenger mobile device 32 is capable of communicating with at least one cloud (e.g., cloud 2) 26. In some embodiments, the passenger mobile device 32 is capable of communicating with the vehicle processing unit 12. Further discussion of the mobile devices 24 and 32 are described below. Other examples of mobile devices 24 and 32 may be found in International Application Publication No. WO2015084353A1, filed Dec. 4, 2013, entitled “Presentation of physiological data,” which describes an example of a user device embodied as a driver mobile device and a passenger mobile device.

In general, the wearable devices 22, 30 may be in wireless communications with the vehicle processing unit 12 and with respective mobile devices 24, 32. In some embodiments, the wearable devices 22, 30 may be in communication with one or both clouds 18, 26, either directly (e.g., via telemetry, such as through a cellular network) or via an intermediate device (e.g., mobile devices 24, 32, respectively). Similarly, the vehicle processing unit 12 may be in communication with one or both clouds 18, 26. In some embodiments, all devices within the vehicle 10 may be in communication with one another and/or with the cloud(s) 18, 26.

The network enabling communications to the clouds 18, 26 may include any of a number of different digital cellular technologies suitable for use in the wireless network, including: GSM, GPRS, CDMAOne, CDMA2000, Evolution-Data Optimized (EV-DO), EDGE, Universal Mobile Telecommunications System (UMTS), Digital Enhanced Cordless Telecommunications (DECT), Digital AMPS (IS-136/TDMA), and Integrated Digital Enhanced Network (iDEN), among others. In some embodiments, communications with devices on the clouds 18, 26 may be achieved using wireless fidelity (WiFi). Access to the clouds 18, 26, which may be part of a wide area network that comprises one or a plurality of networks that in whole or in part comprise the Internet, may be further enabled through access to one or more networks including PSTN (Public Switched Telephone Networks), POTS, Integrated Services Digital Network (ISDN), Ethernet, Fiber, DSL/ADSL, WiFi, Zigbee, BT, BTLE, among others.

Clouds 18, 26 may each comprise an internal cloud, an external cloud, a private cloud, or a public cloud (e.g., commercial cloud). For instance, a private cloud may be implemented using a variety of cloud systems including, for example, Eucalyptus Systems, VMWare vSphere®, or Microsoft® HyperV. A public cloud may include, for example, Amazon EC2®, Amazon Web Services®, Terremark®, Savvis®, or GoGrid®. Cloud-computing resources provided by these clouds may include, for example, storage resources (e.g., Storage Area Network (SAN), Network File System (NFS), and Amazon S3®), network resources (e.g., firewall, load-balancer, and proxy server), internal private resources, external private resources, secure public resources, infrastructure-as-a-services (IaaSs), platform-as-a-services (PaaSs), or software-as-a-services (SaaSs). The cloud architecture may be embodied according to one of a plurality of different configurations. For instance, if configured according to MICROSOFT AZURE™, roles are provided, which are discrete scalable components built with managed code. Worker roles are for generalized development, and may perform background processing for a web role. Web roles provide a web server and listen for and respond to web requests via an HTTP (hypertext transfer protocol) or HTTPS (HTTP secure) endpoint. VM roles are instantiated according to tenant defined configurations (e.g., resources, guest operating system). Operating system and VM updates are managed by the cloud. A web role and a worker role run in a VM role, which is a virtual machine under the control of the tenant. Storage and SQL services are available to be used by the roles. As with other clouds, the hardware and software environment or platform, including scaling, load balancing, etc., are handled by the cloud.

In some embodiments, services of the clouds 18, 26 may be implemented in some embodiments according to multiple, logically-grouped servers (run on server devices), referred to as a server farm. The devices of the server farm may be geographically dispersed, administered as a single entity, or distributed among a plurality of server farms, executing one or more applications on behalf of or in conjunction with one or more of the wearables 22, 30, the mobile devices 24, 32, and/or the vehicle processing unit 12. The devices within each server farm may be heterogeneous. One or more of the devices of the server farm may operate according to one type of operating system platform (e.g., WINDOWS NT, manufactured by Microsoft Corp. of Redmond, Wash.), while one or more of the other devices may operate according to another type of operating system platform (e.g., Unix or Linux). The group of devices of the server farm may be logically grouped as a farm that may be interconnected using a wide-area network (WAN) connection or medium-area network (MAN) connection, and each device may each be referred to as (and operate according to) a file server device, application server device, web server device, proxy server device, or gateway server device.

In some embodiments, the vehicle 10 also includes at least one camera 34. The camera 34 may be located to view the driver's face. In some embodiments, the camera 34 is located to view the passenger's face. In some embodiments, the vehicle 10 may include multiple cameras for viewing the people in the vehicle 10. The camera 34 is capable of communicating with at least one of the vehicle processing unit 12, the wearables 22, 30, the mobile devices 24, 32, and/or the cloud (e.g., cloud 18 and/or cloud 26). In some embodiments, the camera 34 includes a vital signs camera, such as the Philips Vital Signs Camera. The Vital Signs Camera remotely measures heart and breathing rate using a standard, infrared (IR) based camera by sensing changes in skin color and body movement (e.g., chest movement). For instance, whenever the heart beats, the skin color changes because of the extra blood running through the vessels. Algorithms residing within the Vital Signs Camera detect these tiny skin color changes, amplify the signals, and calculate a pulse rate signal by analyzing the frequency of the color changes. For respiration, the Vital Signs Camera focuses on the rise and fall of the chest and/or abdomen, amplifying the signals using algorithms and determining an accurate breathing rate. The Vital Signs Camera is also motion robust, using facial tracking to obtain an accurate reading during motion. The Vital Signs Camera, with its unobtrusive pulse and breathing rate capabilities, enables tracking of moods, sleep patterns, and activity levels, and can be used to help detect driver and/or passenger drowsiness (e.g., sleepiness levels), stress, and attention levels. In general, pulse and breathing rate monitoring are useful when monitoring health, particularly as physiological indicators of emotion. The same or similar functionality may be found in cameras of the wearable devices 22, 30 and/or mobile devices 24, 32.

The driver wearable 22 and/or passenger wearable 30 includes at least one of an accelerometer, photoplethysmograpm (PPG) sensor, sensors for detecting electrodermal activity (EDA) (e.g., detects a variation in the electrical characteristics of the skin, including skin conductance, galvanic skin response, electrodermal response), blood pressure cuff, blood glucose monitor, electrocardiogram sensor, step counter sensor, gyroscope, Sp02 sensor (e.g., providing an estimate of arterial oxygen saturation), respiration sensor, posture sensor, stress sensor, galvanic skin response sensor, temperature sensor, pressure sensor, light sensor, and other physiological parameter sensors. The driver wearable 22 and/or passenger wearable 30 are capable of sensing signals related to heart rate, heart rate variability, respiration rate, pulse transit time, blood pressure, temperature, among other physiological parameters. Other possible parameters and sensors are described in Table 1 of U.S. Pat. No. 8,398,546, filed Sep. 13, 2004, and entitled “System for monitoring and managing body weight and other physiological conditions including iterative and personalized planning, intervention and reporting capability.” In some embodiments, the sensors described above for the driver wearable 22 may be integrated in structures of the vehicle 10 instead (e.g., not worn by the driver 20), yet positioned proximate to the driver 20 in the vehicle 10. For example, the vehicle steering wheel may include one of the sensors (e.g., an ECG sensor). As another example, the driver's seat of the vehicle 10 may include a sensor (e.g., a pressure sensor).

Processing for certain embodiments of the vehicle occupant interaction system may be included in one or any combination of the vehicle processing unit 12, a cloud (e.g., one or more devices of the clouds 18 and/or 26), the driver wearable 22, the passenger wearable 30, the driver mobile device 24, and/or the passenger mobile device 32. Various embodiments of the invention propose to overcome the lack of a way to monitor drivers and passengers and provide updates to such persons regarding the experience or health status of the other person. In the description that follows, primary processing functionality for certain embodiments a vehicle occupant interaction system is described as being achieved in the vehicle processing unit 12, with physiological parameters communicated by the various vehicle sensors 14, 16, wearables 22, 30, camera(s) 34, and/or mobile devices 24 and feedback implemented at various structures within the vehicle 10 (e.g., seats, visual display screens, audio devices, etc.), the wearables 22, 30, and/or the mobile devices 24. It should be appreciated that other devices within or external to the vehicle 10 (e.g., the cloud(s) 18 and/or 26) may be the primary location for processing functionality in some embodiments, and hence are contemplated to be within the scope of the invention.

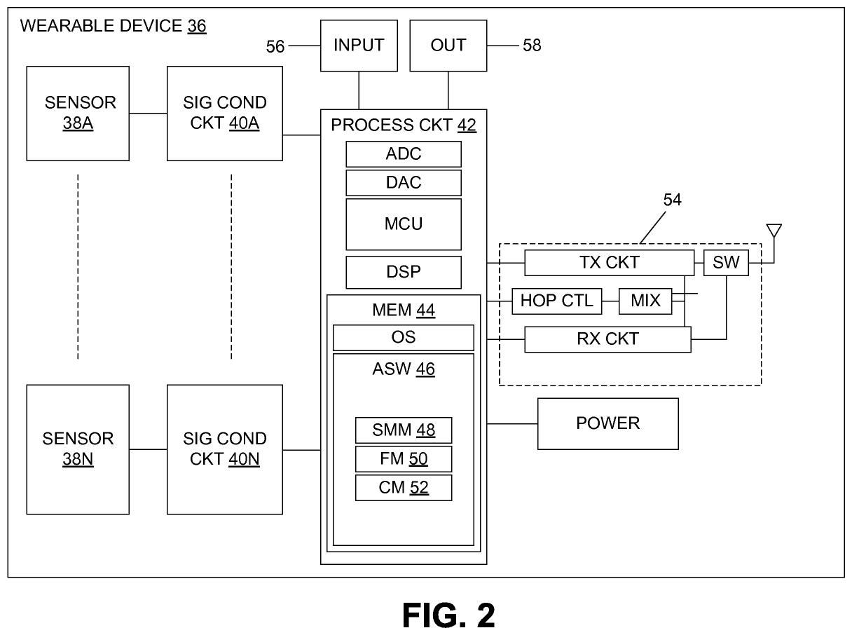

Attention is now directed to FIG. 2, which illustrates an example wearable device 36 in which all or a portion of the functionality of a vehicle occupant interaction system may be implemented. The driver wearable 22 or the passenger wearable 30 may be constructed according to the architecture and functionality of the wearable device 36 depicted in FIG. 2. In particular, FIG. 2 illustrates an example architecture (e.g., hardware and software) for the example wearable device 36. It should be appreciated by one having ordinary skill in the art in the context of the present disclosure that the architecture of the wearable device 36 depicted in FIG. 2 is but one example, and that in some embodiments, additional, fewer, and/or different components may be used to achieve similar and/or additional functionality. In one embodiment, the wearable device 36 comprises a plurality of sensors 38 (e.g., 38A-38N), one or more signal conditioning circuits 40 (e.g., SIG COND CKT 40A-SIG COND CKT 40N) coupled respectively to the sensors 38, and a processing circuit 42 (comprising one or more processors) that receives the conditioned signals from the signal conditioning circuits 40. In one embodiment, the processing circuit 42 comprises an analog-to-digital converter (ADC), a digital-to-analog converter (DAC), a microcontroller unit (MCU), a digital signal processor (DSP), and memory (MEM) 44. In some embodiments, the processing circuit 42 may comprise fewer or additional components than those depicted in FIG. 2. For instance, in one embodiment, the processing circuit 42 may consist entirely of the microcontroller unit. In some embodiments, the processing circuit 42 may include the signal conditioning circuits 40.

The memory 44 comprises an operating system (OS) and application software (ASW) 46, which in one embodiment comprises one or more functionality of a vehicle occupant interaction system. In some embodiments, additional software may be included for enabling physical and/or behavioral tracking, among other functions. In the depicted embodiment, the application software 46 comprises a sensor measurement module (SMM) 48 for processing signals received from the sensors 38, a feedback module (FM) 50 for activating feedback circuitry of the wearable device 36 based on receipt of a control signaling triggering activation (e.g., received in one embodiment, from the vehicle processing unit 12 (FIG. 1), though in some embodiments, feedback may be triggered from other devices or software internal to the wearable device 36), and a communications module (CM) 52. In some embodiments, additional modules used to achieve the disclosed functionality of a vehicle occupant interaction system, among other functionality, may be included, or one or more of the modules 48-52 may be separate from the application software 46 or packaged in a different arrangement than shown relative to each other. In some embodiments, fewer than all of the modules 48-52 may be used in the wearable device 36.

The sensor measurement module 48 comprises executable code (instructions) to process the signals (and associated data) measured by the sensors 38. For instance, the sensors 38 may measure one or more parameters (physiological, emotional, etc.) including heart rate, heart rate variability, electrodermal activity, and/or body motion (e.g., using single or tri-axial accelerometer measurements). One or more of these parameters may be analyzed by the sensor measurement module 48, enabling a derivation of indicators of the health and/or well-being of the subject wearing the wearable device 36, including indicators of stress, indicators of anxiety, indicators of motion sickness, sleepiness, etc. In some embodiments, the raw data corresponding to one or more of the parameters is communicated to the vehicle processing unit 12 (FIG. 1), which derives the indicators from the raw data. In some embodiments, functionality of the sensor measurement module 48 may be achieved locally and at other devices (e.g., the vehicle processing unit 12) in distributed computing fashion. The sensor measurement module 48 may control the sampling rate of one or more of the sensors 38.

The feedback module 50 comprises executable code (instructions) to receive a triggering signal and activate feedback circuitry. In one embodiment, the triggering signal may be communicated from another device within the vehicle 10 (FIG. 1), including from another wearable, a mobile device, or the vehicle processing unit 12 (FIG. 1). In some embodiments, the application software 46 of the wearable device 36, for instance where processing functionality for determining passenger or driver stress and/or frustration, sleepiness, and/or suitable nap/inattentive times is achieved by the wearable device 36, communicates the triggering signal. In some embodiments, the triggering signal is communicated from other devices within the vehicle 10 (FIG. 1) (e.g., the camera 34), or external to the vehicle 10 (e.g., from a device of the cloud(s) 18, 26). The feedback module 50, based on receiving the triggering signal, activates one or more circuitry of the wearable device 36. For instance, a vibratory motor in the wearable device 36 may be activated to haptically alert the possessor of the wearable device 36 of, say, passenger stress (or driver stress when worn by the passenger) or sleepiness. As another example, lighting (e.g., light emitting diode or LED) of the wearable device 36 may be activated to visibly alert the possessor of the wearable device 36, or audio circuitry (e.g., a buzzer) may be activated to audibly alert the possessor of the wearable device 36. In some embodiments, the wearable device 36 may comprise a display screen, where an alert is presented in the form of text or graphical messages, or lighting. In some embodiments, any combination of the tactile, visual, or audible feedback may be implemented. In some embodiments, the feedback may be modulated in intensity depending on the triggering signal. For instance, the frequency of LED light activation (e.g., blinking lights) or display lighting may be changed depending on the emotional, behavioral, or physiological state or condition of the monitored subject. In the case of monitoring physiological or emotional state of the passenger, for instance, the trigger signal from the vehicle processing unit 12, for instance, may be modulated in a manner to reflect the intensity of the stress levels, which may be manifested in the feedback via a more rapid blinking of lighting as stress increases, or increased beep frequency or volume, or increased strength of the vibration, or transition to other feedback mechanisms (e.g., buzzer to beep, beep to visual stimuli, etc.), among other forms of feedback. In general, the feedback may be adjusted in a manner to reflect the sensed health or well-being changes to enable a discrimination in levels of changes in some embodiments. In some embodiments, the wearable device 36 may communicate a signal to another device for activation of feedback mechanisms in that other device.

The communications module 52 comprises executable code (instructions) to enable a communications circuit 54 of the wearable device 36 to operate according to one or more of a plurality of different communication technologies (e.g., NFC, Bluetooth, Zigbee, 802.11, Wireless-Fidelity, GSM, etc.) to receive from, and/or transmit data to, one or more devices (e.g., other wearable devices, mobile devices, cloud devices, vehicle processing unit, cameras, etc.) internal to the vehicle 10 or external to the vehicle 10. For purposes of illustration, the communications module 52 is described herein as providing for control of communications with the vehicle processing unit 12 (FIG. 1). In one embodiment, one or more sensed parameters are communicated to the vehicle processing unit 12 via the communications circuit 54 in conjunction with the communications module 52, and triggering signals are received from the vehicle processing unit 12 via the communications circuit 54 in conjunction with the communications module 52. As explained above, the parameters communicated to the vehicle processing unit 12 may be raw data or derived data, or a combination of both.

As indicated above, in one embodiment, the processing circuit 42 is coupled to the communications circuit 54. The communications circuit 54 serves to enable wireless communications between the wearable device 36 and other devices within or external to the vehicle 10 (FIG. 1). The communications circuit 54 is depicted as a Bluetooth (BT) circuit, though not limited to this transceiver configuration. For instance, in some embodiments, the communications circuit 54 may be embodied as any one or a combination of an NFC circuit, Wi-Fi circuit, transceiver circuitry based on Zigbee, BT low energy, 802.11, GSM, LTE, CDMA, WCDMA, among others such as optical or ultrasonic based technologies. In some embodiments, plural transceiver circuits according to more than one of the communication specifications/standards described above may be used.

The processing circuit 42 is further coupled to input/output (I/O) devices or peripherals, including an input interface 56 (INPUT) and an output interface 58 (OUT). In some embodiments, an input interface 56 and/or output interface 58 may be omitted, or functionality of both may be combined into a single component.

Note that in some embodiments, functionality for one or more of the aforementioned circuits and/or software may be combined into fewer components/modules, or in some embodiments, further distributed among additional components/modules or devices. For instance, the processing circuit 42 may be packaged as an integrated circuit that includes the microcontroller (microcontroller unit or MCU), the DSP, and memory 44, whereas the ADC and DAC may be packaged as a separate integrated circuit coupled to the processing circuit 42. In some embodiments, one or more of the functionality for the above-listed components may be combined, such as functionality of the DSP performed by the microcontroller.

As indicated above, the sensors 38 comprise one or any combination of sensors capable of measuring physiological, emotional, and/or behavioral parameters. For instance, typical physiological parameters include heart rate, heart rate variability, heart rate recovery, blood flow rate, activity level, muscle activity (including core movement, body orientation/position, power, speed, acceleration, etc.), muscle tension, blood volume, blood pressure, blood oxygen saturation, respiratory rate, perspiration, skin temperature, electrodermal activity (skin conductance response, galvanic skin response, electrodermal response, etc.), body weight, and body composition (e.g., body mass index or BMI), articulator movements (especially during speech), iris scans (e.g., using imaging sensors). The physiological parameters may be used to determine various information. For instance, typical behavioral information includes various sleep level behavior, vehicle driving style behavior (e.g., using an accelerometer sensor to measure or detect rapid, irregular steering wheel movement and/or hand position on the steering wheel, foot movement (e.g., movement on the brake or accelerator pedals), shifting movement by the hand, etc. Note that in some embodiments, vehicular sensors may provide for a similar characterization of driving style. Other information includes driving location (e.g., using global navigation satellite system (GNSS) sensors/receiver), including start and end points and route(s) in between. As another example, emotional information may be gathered based on the physiological information, including stress, anxiety. Such indicators may include pupil dilation or other facial feature changes, heart rate, voice pattern and/or volume, gesture sensing, breathing rate, among others. Other information may include sickness, such as motion sickness. The sensors 38 may also include inertial sensors (e.g., gyroscopes) and/or magnetometers, which may assist in the determination of driving behavior and correlation with motion sickness, for instance. In some embodiments, as indicated above, the sensors 38 may include GNSS sensors, including a GPS receiver to facilitate determinations of distance, speed, acceleration, location, altitude, etc. (e.g., location data, or generally, sensing movement). In some embodiments, GNSS sensors (e.g., GNSS receiver and antenna(s)) may be included in the mobile device(s) 24, 32 (FIG. 1) and/or the vehicle 10 (FIG. 1), in addition to, or in lieu of, those residing in the wearable device 36. In some embodiments, GNSS functionality may be achieved via the communications circuit 54 or other circuits coupled to the processing circuit 42. The sensors 38 may also include flex and/or force sensors (e.g., using variable resistance), electromyographic sensors, electrocardiographic sensors (e.g., EKG, ECG), magnetic sensors, photoplethysmographic (PPG) sensors, bio-impedance sensors, infrared proximity sensors, acoustic/ultrasonic/audio sensors, a strain gauge, galvanic skin/sweat sensors, pH sensors, temperature sensors, and photocells. The sensors 38 may include other and/or additional types of sensors for the detection of environmental parameters and/or conditions, for instance, barometric pressure, humidity, outdoor temperature, pollution, noise level, etc. One or more of these sensed environmental parameters/conditions may be influential in the determination of the state of the user. Note that one or more of the sensors 38 may be constructed based on piezoelectric, piezoresistive or capacitive technology in a microelectromechanical system (MEMS) infrastructure.

The signal conditioning circuits 40 include amplifiers and filters, among other signal conditioning components, to condition the sensed signals including data corresponding to the sensed physiological parameters and/or location signals before further processing is implemented at the processing circuit 42. Though depicted in FIG. 2 as respectively associated with each sensor 38, in some embodiments, fewer signal conditioning circuits 40 may be used (e.g., shared for more than one sensor 38). In some embodiments, the signal conditioning circuits 40 (or functionality thereof) may be incorporated elsewhere, such as in the circuitry of the respective sensors 38 or in the processing circuit 42 (or in components residing therein). Further, although described above as involving unidirectional signal flow (e.g., from the sensor 38 to the signal conditioning circuit 40), in some embodiments, signal flow may be bi-directional. For instance, in the case of optical measurements, the microcontroller may cause an optical signal to be emitted from a light source (e.g., light emitting diode(s) or LED(s)) in or coupled to the circuitry of the sensor 38, with the sensor 38 (e.g., photocell) receiving the reflected/refracted signals.

The communications circuit 54 is managed and controlled by the processing circuit 42 (e.g., executing the communications module 52). The communications circuit 54 is used to wirelessly interface with the vehicle processing unit 12 (FIG. 1) and/or in some embodiments, one or more devices within and/or external to the vehicle 10 (FIG. 1). In one embodiment, the communications circuit 54 may be configured as a Bluetooth transceiver, though in some embodiments, other and/or additional technologies may be used, such as Wi-Fi, GSM, LTE, CDMA and its derivatives, Zigbee, NFC, among others. In the embodiment depicted in FIG. 2, the communications circuit 54 comprises a transmitter circuit (TX CKT), a switch (SW), an antenna, a receiver circuit (RX CKT), a mixing circuit (MIX), and a frequency hopping controller (HOP CTL). The transmitter circuit and the receiver circuit comprise components suitable for providing respective transmission and reception of an RF signal, including a modulator/demodulator, filters, and amplifiers. In some embodiments, demodulation/modulation and/or filtering may be performed in part or in whole by the DSP. The switch switches between receiving and transmitting modes. The mixing circuit may be embodied as a frequency synthesizer and frequency mixers, as controlled by the processing circuit 42. The frequency hopping controller controls the hopping frequency of a transmitted signal based on feedback from a modulator of the transmitter circuit. In some embodiments, functionality for the frequency hopping controller may be implemented by the microcontroller or DSP. Control for the communications circuit 54 may be implemented by the microcontroller, the DSP, or a combination of both. In some embodiments, the communications circuit 54 may have its own dedicated controller that is supervised and/or managed by the microcontroller.

In one example operation for the communications circuit 54, a signal (e.g., at 2.4 GHz) may be received at the antenna and directed by the switch to the receiver circuit. The receiver circuit, in cooperation with the mixing circuit, converts the received signal into an intermediate frequency (IF) signal under frequency hopping control attributed by the frequency hopping controller and then to baseband for further processing by the ADC. On the transmitting side, the baseband signal (e.g., from the DAC of the processing circuit 42) is converted to an IF signal and then RF by the transmitter circuit operating in cooperation with the mixing circuit, with the RF signal passed through the switch and emitted from the antenna under frequency hopping control provided by the frequency hopping controller. The modulator and demodulator of the transmitter and receiver circuits may perform frequency shift keying (FSK) type modulation/demodulation, though not limited to this type of modulation/demodulation, which enables the conversion between IF and baseband. In some embodiments, demodulation/modulation and/or filtering may be performed in part or in whole by the DSP. The memory 44 stores the communications module 52, which when executed by the microcontroller, controls the Bluetooth (and/or other protocols) transmission/reception.

Though the communications circuit 54 is depicted as an IF-type transceiver, in some embodiments, a direct conversion architecture may be implemented. As noted above, the communications circuit 54 may be embodied according to other and/or additional transceiver technologies.

The processing circuit 42 is depicted in FIG. 2 as including the ADC and DAC. For sensing functionality, the ADC converts the conditioned signal from the signal conditioning circuit 40 and digitizes the signal for further processing by the microcontroller and/or DSP. The ADC may also be used to convert analogs inputs that are received via the input interface 56 to a digital format for further processing by the microcontroller. The ADC may also be used in baseband processing of signals received via the communications circuit 54. The DAC converts digital information to analog information. Its role for sensing functionality may be to control the emission of signals, such as optical signals or acoustic signals, from the sensors 38. The DAC may further be used to cause the output of analog signals from the output interface 58. Also, the DAC may be used to convert the digital information and/or instructions from the microcontroller and/or DSP to analog signals that are fed to the transmitter circuit. In some embodiments, additional conversion circuits may be used.

The microcontroller and the DSP provide processing functionality for the wearable device 36. In some embodiments, functionality of both processors may be combined into a single processor, or further distributed among additional processors. The DSP provides for specialized digital signal processing, and enables an offloading of processing load from the microcontroller. The DSP may be embodied in specialized integrated circuit(s) or as field programmable gate arrays (FPGAs). In one embodiment, the DSP comprises a pipelined architecture, which comprises a central processing unit (CPU), plural circular buffers and separate program and data memories according to a Harvard architecture. The DSP further comprises dual busses, enabling concurrent instruction and data fetches. The DSP may also comprise an instruction cache and I/O controller, such as those found in Analog Devices SHARC® DSPs, though other manufacturers of DSPs may be used (e.g., Freescale multi-core MSC81xx family, Texas Instruments C6000 series, etc.). The DSP is generally utilized for math manipulations using registers and math components that may include a multiplier, arithmetic logic unit (ALU, which performs addition, subtraction, absolute value, logical operations, conversion between fixed and floating point units, etc.), and a barrel shifter. The ability of the DSP to implement fast multiply-accumulates (MACs) enables efficient execution of Fast Fourier Transforms (FFTs) and Finite Impulse Response (FIR) filtering. Some or all of the DSP functions may be performed by the microcontroller. The DSP generally serves an encoding and decoding function in the wearable device 36. For instance, encoding functionality may involve encoding commands or data corresponding to transfer of information. Also, decoding functionality may involve decoding the information received from the sensors 38 (e.g., after processing by the ADC).

The microcontroller comprises a hardware device for executing software/firmware, particularly that stored in memory 44. The microcontroller can be any custom made or commercially available processor, a central processing unit (CPU), a semiconductor based microprocessor (in the form of a microchip or chip set), a macroprocessor, or generally any device for executing software instructions. Examples of suitable commercially available microprocessors include Intel's® Itanium® and Atom® microprocessors, to name a few non-limiting examples. The microcontroller provides for management and control of the wearable device 36.

The memory 44 (also referred to herein as a non-transitory computer readable medium) can include any one or a combination of volatile memory elements (e.g., random access memory (RAM, such as DRAM, SRAM, SDRAM, etc.)) and nonvolatile memory elements (e.g., ROM, Flash, solid state, EPROM, EEPROM, etc.). Moreover, the memory 44 may incorporate electronic, magnetic, and/or other types of storage media. The memory 44 may be used to store sensor data over a given time duration and/or based on a given storage quantity constraint for later processing.

The software in memory 44 may include one or more separate programs, each of which comprises an ordered listing of executable instructions for implementing logical functions. In the example of FIG. 2, the software in the memory 44 includes a suitable operating system and the application software 46, which in one embodiment, comprises sensor measurement, feedback generating, and communications capabilities via modules 48, 50, and 52, respectively.

The operating system essentially controls the execution of computer programs, such as the application software 46 and associated modules 48-52, and provides scheduling, input-output control, file and data management, memory management, and communication control and related services. The memory 44 may also include user data, including weight, height, age, gender, goals, body mass index (BMI) that may be used by the microcontroller executing executable code to accurately interpret the measured parameters. The user data may also include historical data relating past recorded data to prior contexts, including sleep history, In some embodiments, user data may be stored elsewhere (e.g., at the mobile devices 24, 32 (FIG. 1), the vehicle processing unit 12 (FIG. 1), or remotely (e.g., in a storage device in the cloud(s) 18, 26 (FIG. 1).

The software in memory 44 comprises a source program, executable program (object code), script, or any other entity comprising a set of instructions to be performed. When a source program, then the program may be translated via a compiler, assembler, interpreter, or the like, so as to operate properly in connection with the operating system. Furthermore, the software can be written as (a) an object oriented programming language, which has classes of data and methods, or (b) a procedure programming language, which has routines, subroutines, and/or functions, for example but not limited to, C, C++, Python, Java, among others. The software may be embodied in a computer program product, which may be a non-transitory computer readable medium or other medium.

The input interface(s) 56 comprises one or more interfaces (e.g., including a user interface) for entry of user input, such as a button or microphone or sensor(s) (e.g., to detect user input, including as a touch-type display screen). In some embodiments, the input interface 56 may serve as a communications port for downloaded information to the wearable device 36 (such as via a wired connection). The output interface(s) 58 comprises one or more interfaces for presenting feedback or data transfer (e.g., wired), including a user interface (e.g., display screen presenting a graphical or other type of user interface, virtual or augmented reality interface, etc.) or communications interface for the transfer (e.g., wired) of information stored in the memory 44. The output interface 58 may comprise other types of feedback devices, such as lighting devices (e.g., LEDs), audio devices (e.g., tone generator and speaker), and/or tactile feedback devices (e.g., vibratory motor) and/or electrical feedback devices.

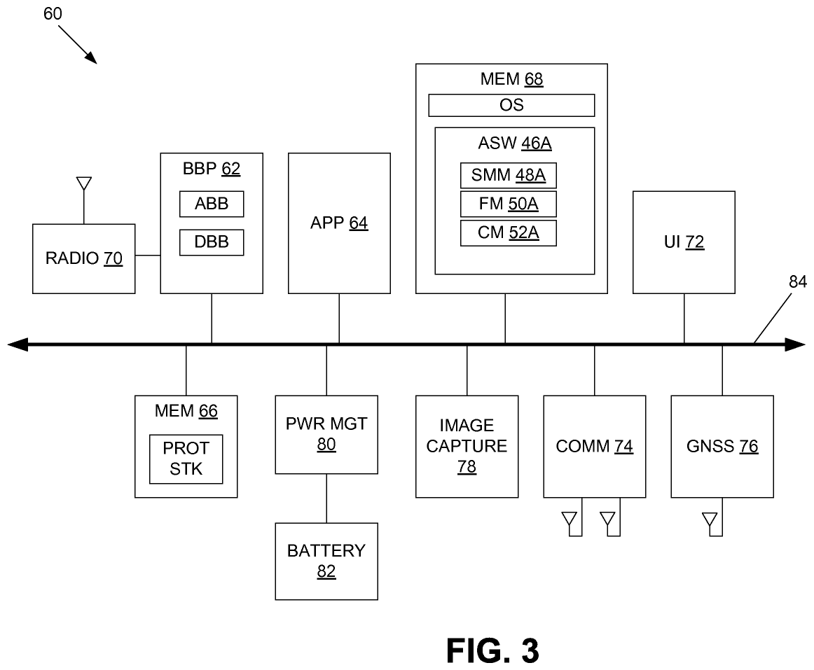

Referring now to FIG. 3, shown is an example mobile device 60 in which all or a portion of the functionality of a vehicle occupant interaction system may be implemented. The driver mobile device 24 and the passenger mobile device 32 may each be constructed according to the architecture and functionality of the mobile device 60 depicted in FIG. 3. In particular, FIG. 3 illustrates an example architecture (e.g., hardware and software) for the example mobile device 60. It should be appreciated by one having ordinary skill in the art in the context of the present disclosure that the architecture of the mobile device 60 depicted in FIG. 3 is but one example, and that in some embodiments, additional, fewer, and/or different components may be used to achieve similar and/or additional functionality. In the depicted example, the mobile device 60 is embodied as a smartphone, though in some embodiments, other types of devices may be used, including a workstation, laptop, notebook, tablet, etc. The mobile device 60 may be used in some embodiments to provide the entire functionality of certain embodiments of a vehicle occupant interaction system, or in some embodiments, provide functionality of the vehicle occupant interaction system in conjunction with one or any combination of the wearable device 36 (FIG. 2), the vehicle processing unit 12 (FIG. 1), or one or more devices of the cloud(s) 18, 26 (FIG. 1). In the description that follows, the mobile device 60 is described as providing parameter sensing, feedback, and communications functionality, similar to that described for the wearable device 36, with the understanding that the mobile device 60 may provide fewer or greater functionality of the vehicle occupant interaction system in some embodiments.

The mobile device 60 comprises at least two different processors, including a baseband processor (BBP) 62 and an application processor (APP) 64. As is known, the baseband processor 62 primarily handles baseband communication-related tasks and the application processor 64 generally handles inputs and outputs and all applications other than those directly related to baseband processing. The baseband processor 62 comprises a dedicated processor for deploying functionality associated with a protocol stack (PROT STK), such as but not limited to a GSM (Global System for Mobile communications) protocol stack, among other functions. The application processor 64 comprises a multi-core processor for running applications, including all or a portion of application software 46A. The baseband processor 62 and the application processor 64 have respective associated memory (e.g., MEM) 66, 68, including random access memory (RAM), Flash memory, etc., and peripherals, and a running clock. The memory 66, 68 are each also referred to herein as a non-transitory computer readable medium. Note that, though depicted as residing in memory 68, all or a portion of the modules of the application software 46A may be stored in memory 66, distributed among memory 66, 68, or reside in other memory.

The baseband processor 62 may deploy functionality of the protocol stack to enable the mobile device 60 to access one or a plurality of wireless network technologies, including WCDMA (Wideband Code Division Multiple Access), CDMA (Code Division Multiple Access), EDGE (Enhanced Data Rates for GSM Evolution), GPRS (General Packet Radio Service), Zigbee (e.g., based on IEEE 802.15.4), Bluetooth, Wi-Fi (Wireless Fidelity, such as based on IEEE 802.11), and/or LTE (Long Term Evolution), among variations thereof and/or other telecommunication protocols, standards, and/or specifications. The baseband processor 62 manages radio communications and control functions, including signal modulation, radio frequency shifting, and encoding. The baseband processor 62 comprises, or may be coupled to, a radio (e.g., RF front end) 70 and/or a GSM (or other communications standard) modem, and analog and digital baseband circuitry (ABB, DBB, respectively in FIG. 3). The radio 70 comprises one or more antennas, a transceiver, and a power amplifier to enable the receiving and transmitting of signals of a plurality of different frequencies, enabling access to a cellular (and/or wireless) network. The analog baseband circuitry is coupled to the radio 70 and provides an interface between the analog and digital domains of the GSM modem. The analog baseband circuitry comprises circuitry including an analog-to-digital converter (ADC) and digital-to-analog converter (DAC), as well as control and power management/distribution components and an audio codec to process analog and/or digital signals received indirectly via the application processor 64 or directly from a user interface (UI) 72 (e.g., microphone, earpiece, ring tone, vibrator circuits, touch-screen, etc.). The ADC digitizes any analog signals for processing by the digital baseband circuitry. The digital baseband circuitry deploys the functionality of one or more levels of the GSM protocol stack (e.g., Layer 1, Layer 2, etc.), and comprises a microcontroller (e.g., microcontroller unit or MCU, also referred to herein as a processor) and a digital signal processor (DSP, also referred to herein as a processor) that communicate over a shared memory interface (the memory comprising data and control information and parameters that instruct the actions to be taken on the data processed by the application processor 64). The MCU may be embodied as a RISC (reduced instruction set computer) machine that runs a real-time operating system (RTIOS), with cores having a plurality of peripherals (e.g., circuitry packaged as integrated circuits) such as RTC (real-time clock), SPI (serial peripheral interface), I2C (inter-integrated circuit), UARTs (Universal Asynchronous Receiver/Transmitter), devices based on IrDA (Infrared Data Association), SD/MMC (Secure Digital/Multimedia Cards) card controller, keypad scan controller, and USB devices, GPRS crypto module, TDMA (Time Division Multiple Access), smart card reader interface (e.g., for the one or more SIM (Subscriber Identity Module) cards), timers, and among others. For receive-side functionality, the MCU instructs the DSP to receive, for instance, in-phase/quadrature (I/Q) samples from the analog baseband circuitry and perform detection, demodulation, and decoding with reporting back to the MCU. For transmit-side functionality, the MCU presents transmittable data and auxiliary information to the DSP, which encodes the data and provides to the analog baseband circuitry (e.g., converted to analog signals by the DAC).

The application processor 64 operates under control of an operating system (OS) that enables the implementation of a plurality of user applications, including the application software 46A. The application processor 64 may be embodied as a System on a Chip (SOC), and supports a plurality of multimedia related features including web browsing/cloud-based access functionality to access one or more computing devices, of the cloud(s) 18, 26 (FIG. 1), that are coupled to the Internet. For instance, the application processor 64 may execute communications functionality of the application software 46A (e.g., middleware, similar to some embodiments of the wearable device 36, which may include a browser with or operable in association with one or more application program interfaces (APIs)) to enable access to a cloud computing framework or other networks to provide remote data access/storage/processing, and through cooperation with an embedded operating system, access to calendars, location services, user data, public data, etc. For instance, in some embodiments, the vehicle occupant interaction system may operate using cloud computing services, where the processing of raw and/or derived parameter data received, indirectly via the mobile device 60 or directly from the wearable device 36 or the vehicle processing unit 12, FIG. 1) may be achieved by one or more devices of the cloud(s) 18, 26 (FIG. 1), and triggering signals (to trigger feedback) may be communicated from the cloud(s) 18, 26 (or other devices) to the mobile device 60, which in turn may activate feedback internal to the mobile device 60 (e.g., visually, audibly, or via tactile mechanisms) or relay the triggering signals to other devices (e.g., the wearable device 36 and/or the vehicle processing unit 12). In the depicted example, the application software 46A relies on processing by the vehicle processing unit 12 based on the sensing of physiological parameters by the mobile device 60 (and communication of the same to the vehicle processing unit 12), and responds to trigger signals sent by the vehicle processing unit 12 to activate one or more types of feedback functionality at the mobile device 60, with the understanding that additional and/or different processing may occur at the mobile device 60 in some embodiments. The application processor 64 generally comprises a processor core (Advanced RISC Machine or ARM), and further comprises or may be coupled to multimedia modules (for decoding/encoding pictures, video, and/or audio), a graphics processing unit (GPU), communications interface (COMM) 74, and device interfaces. In one embodiment, the communications interfaces 74 may include wireless interfaces, including a Bluetooth (BT) (and/or Zigbee in some embodiments, among others) module that enable wireless communication with the wearable device 36, other mobile devices, and/or the vehicle processing unit 12. In some embodiments, the communications interface 74 may comprise a Wi-Fi module for interfacing with a local 802.11 network, according to corresponding communications software in the applications software 46A. The application processor 64 further comprises, or in the depicted embodiment, is coupled to, a global navigation satellite systems (GNSS) receiver 76 for enabling access to a satellite network to, for instance, provide position coordinates. In some embodiments, the GNSS receiver 76, in association with GNSS functionality in the application software 46A, collects contextual data (time and location data, including location coordinates and altitude) to help establish a pattern of behavior (in conjunction with sensing functionality), for instance when the driver and/or passenger possessing the mobile device 60 are away from the vehicle. Note that, though described as a GNSS receiver 76, other indoor/outdoor positioning systems may be used, including those based on triangulation of cellular network signals and/or Wi-Fi.

The device interfaces coupled to the application processor 64 may include the user interface 72, including a display screen. The display screen, in some embodiments similar to a display screen of the wearable device user interface, may be embodied in one of several available technologies, including LCD or Liquid Crystal Display (or variants thereof, such as Thin Film Transistor (TFT) LCD, In Plane Switching (IPS) LCD)), light-emitting diode (LED)-based technology, such as organic LED (OLED), Active-Matrix OLED (AMOLED), retina or haptic-based technology, or virtual/augmented reality technology. For instance, the user interface 72 may present visual feedback in the form of messaging (e.g., text messages) and/or symbols/graphics (e.g., warning or alert icons, flashing screen, etc.), and/or flashing lights (LEDs). In some embodiments, the user interface 72 may be configured, in addition to or in lieu of a display screen, a keypad, microphone, speaker, ear piece connector, I/O interfaces (e.g., USB (Universal Serial Bus)), SD/MMC card, among other peripherals. For instance, the speaker may be used to audibly provide feedback, and/or the user interface 72 may comprise a vibratory motor that provides a vibrating feedback to the user. One or any combination of visual, audible, or tactile feedback may be used, and as described before, variations in the intensity of format of the feedback may be used to provide levels of a given health condition and/or emotion (e.g., increasingly stressed) as indicated by, say, a different color (e.g., red) than initial stress levels (e.g., yellow) when presented on the display screen.

Also coupled to the application processor 64 is an image capture device (IMAGE CAPTURE) 78. The image capture device 78 comprises an optical sensor (e.g., a charged coupled device (CCD) or a complementary metal-oxide semiconductor (CMOS) optical sensor). In one embodiment, the image capture device 78 may be configured as a Vital Signs Camera, as described above. In general, the image capture device 78 may be used to detect various physiological parameters of a user, including blood pressure (e.g., based on remote photoplethysmography (PPG)), heart rate, and/or breathing patterns. Also included is a power management device 80 that controls and manages operations of a battery 82. The components described above and/or depicted in FIG. 3 share data over one or more busses, and in the depicted example, via data bus 84. It should be appreciated by one having ordinary skill in the art, in the context of the present disclosure, that variations to the above may be deployed in some embodiments to achieve similar functionality.

In the depicted embodiment, the application processor 64 runs the application software 46A, which comprises a sensor measurement module 48A, a feedback module 50A, and a communications module 52A. The sensor measurement module 48A receives physiological parameters and/or contextual data (e.g., location data) from sensors of the mobile device 60, including from the image capture device 78 and GNSS receiver 76, respectively. The feedback module 50A provides for visual, audible, and or tactile feedback to the user via the UI 72. The communications module 52A communicates raw and/or derived parameters to one or more other devices located within or external to the vehicle 10, and also receives triggering signals to activate the feedback functionality. For instance, in one embodiment, the mobile device 60 communicates parameters to the vehicle processing unit 12 (FIG. 1), and receives from the vehicle processing unit 12 triggering signals that the feedback module 50A, in conjunction with the user interface 72, provides feedback to the user (e.g., driver or passenger). These modules 48A, 50A, and 54A of the applications software 46A are similar to like-numbered modules of the application software 46 described in association with FIG. 2, and hence further description of the same is omitted here for brevity.

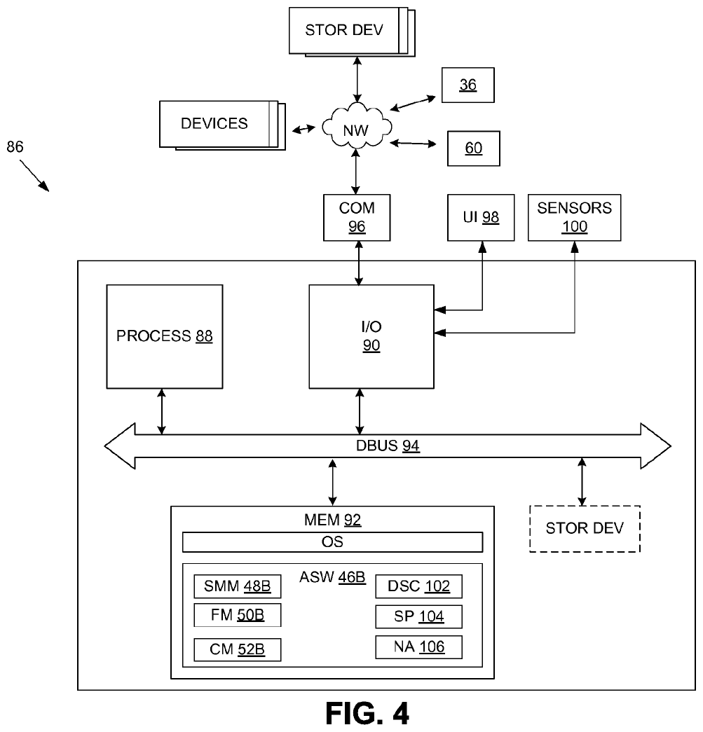

Referring now to FIG. 4, shown is an embodiment of an example vehicle processing unit 86 in which in which all or a portion of the functionality of a vehicle occupant interaction system may be implemented. The vehicle processing unit 12 (FIG. 1) may comprise the functionality and structure of the vehicle processing unit 86 depicted in FIG. 4. Functionality of the vehicle processing unit 86 may be implemented alone, or in some embodiments, in combination with one or more additional devices. In one embodiment, the vehicle processing unit 86 may be embodied as a computer, though in some embodiments, may be embodied as an application server (e.g., if functionality of the vehicle occupant interaction system is implemented primarily remotely). One having ordinary skill in the art should appreciate in the context of the present disclosure that the example vehicle processing unit 86 is merely illustrative of one embodiment, and that some embodiments may comprise fewer or additional components. The vehicle processing unit 86 is depicted in this example as a computer system. It should be appreciated that certain well-known components of computer systems are omitted here to avoid obfuscating relevant features of the vehicle processing unit 86. In one embodiment, the vehicle processing unit 86 comprises hardware and software components, including one or more processors (one shown), such as processor (PROCESS) 88, input/output (I/O) interface(s) 90 (I/O), and memory 92 (MEM), all coupled to one or more data busses, such as data bus 94 (DBUS). The memory 92 (also referred to herein as a non-transitory computer readable medium) may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.). The memory 92 may store a native operating system (OS), one or more native applications, emulation systems, or emulated applications for any of a variety of operating systems and/or emulated hardware platforms, emulated operating systems, etc. In some embodiments, one or more separate storage devices (STOR DEV) may be coupled to the data bus 94 and/or the vehicle processing unit 86 may be coupled to network storage via a network and communications functionality as described further below.

In the depicted embodiment, the vehicle processing unit 86 is coupled via the I/O interfaces 90 to a communications interface (COM) 96, a user interface (UI) 98, and one or more sensors 100. In some embodiments, the communications interface 96, user interface 98, and one or more sensors 100 may be coupled directly to the data bus 94. The communications interface 96 comprises hardware and software for wireless functionality (e.g., Bluetooth, near field communications, Wi-Fi, etc.), enabling wireless communications with devices located internal to the vehicle 10 (FIG. 1), including the wearable device 36 (FIG. 2), mobile device 60 (FIG. 3), among other devices (e.g., camera 34), and optionally wireless communications with sensors 100 of the vehicle 10 that are located on the exterior of the vehicle 10. In some embodiments, wireless communications may be enabled via the communications interface 96 between the vehicle processing unit 86 and mobile devices and/or wearable devices in other nearby vehicles. In one embodiment, the communications interface 96 further comprises cellular modem functionality to enable cellular communications to access computing functionality of the cloud(s) 18, 26 (FIG. 1), such as to access public or proprietary data structures (e.g., databases). For instance, a user profile may be located in one or more devices of the cloud(s) 18, 26, and includes user data (e.g., age, gender, sleep history, activity history, etc. of the driver and/or passenger) and/or public statistics (e.g., road information, including traffic statistics (e.g., via WAZE or DOT web-sites), road geography/topology, injury/death statistics for the road(s), construction information, weather data, mapping/location services, etc.). In some embodiments, the weather data may be acquired via sensors located within (or located on the exterior of the vehicle 10), or via stand-alone devices found within the vehicle 10, including through the use of a netamo device. In some embodiments, one or more of the information may be stored locally for a transitory period (e.g., in storage device and/or memory 92).

The I/O interfaces 90 may comprise any number of interfaces for the input and output of signals (e.g., analog or digital data) for conveyance of information (e.g., data) over various networks and according to various protocols and/or standards.

The user interface 98 comprises one or any combination of a display screen with or without a graphical user interface (GUI), heads-up display, keypad, vehicle buttons/switches/knobs or other mechanisms to enable the entry of user commands for the vehicle controls, microphone, mouse, etc., and/or feedback to the driver and/or passenger. For instance, the user interface 98 may include dedicated lighting (e.g., internal status lights, such as a warning light or caution light or pattern) or other mechanisms to provide visual feedback, including a console display having emoji icons or other symbolic graphics or even text warning of passenger sentiment or sleep state. In some embodiments, the user interface 98 comprises one or more vibratory motors (e.g., in the driver and/or passenger seat, stick-shift, steering wheel, arm rest, etc.) to provide tactile feedback to the driver and/or passenger within the vehicle 10 (FIG. 1), such as to warn the passenger of driver sentiment (e.g., if behavior by the passenger is aggravating or stressing the driver, or if the driver is getting sleepy) or to warn the driver (e.g., if the driver's style of driving is causing stress or sickness to the passenger, or if the passenger is falling asleep when the driver needs the passenger to be attentive). In some embodiments, the user interface 98 comprises speakers and/or microphones, such as to provide verbal beeping or other sounds (e.g., tones, or verbal speaking) that warn of the aforementioned driver and/or passenger states or conditions. The intensity of the various feedback may also be altered, such as increasing frequency or volume of sounds as the condition worsens (e.g., as the motion sickness of the passenger gets worse, the warning to the user is increased in frequency and/or intensity). In some embodiments, the device used to present the feedback may be changed based on the parameter intensity. Note that one or any combination of the various feedback techniques and/or devices described above may be used at any one time.

The sensors 100 comprise internal and external sensors (e.g., internal sensors 16 and external sensor 14, FIG. 1), including camera sensors (e.g., camera 34, FIG. 1) and/or position locating sensors (e.g., GNSS receiver). The sensors 100 include the vehicle sensors that are associated with vehicle motion, including inertial motion sensors (e.g., gyroscopes, magnetometers), load sensors, position sensors, velocity sensors, and/or acceleration sensors. In other words, the sensors 100 measure the vehicle movement information associated with the driver's style of driving, including the abruptness of starts and stops, fast accelerations, speed, sharp turns, and/or odd movements.

In the embodiment depicted in FIG. 4, the memory 92 comprises an operating system (OS) and application software (ASW) 46B. Note that in some embodiments, the application software 46B may be implemented without the operating system. In one embodiment, the application software 46B comprises a sensor measurement module 48B, a feedback module 50B, a communications module 52B, a driving style correlator (DSC) module 102, a sleepiness prediction (SP) module 104, and a nap/alertness (NA) module 106. The sensor measurement module 48B receives raw or derived parameters from the sensors 100 (and/or from other devices located within the vehicle 10 (FIG. 1) and/or external to the vehicle 10 via the communications module 52B and formats for use in the modules 102-106. In some embodiments, such functionality may be located in other devices configured to provide the data in a useable format in the vehicle processing unit, and hence may be omitted from the application software 46B in some embodiments. More generally, some embodiments may combine the aforementioned functionality or further distribute the functionality among additional modules and/or devices. In some embodiments, the data is provided to the feedback module 50B for providing feedback to one of the occupants of the vehicle 10 (e.g., via the user interface 98) and/or in combination with the communications module 52B for providing feedback to occupants of one or more other vehicles. For instance, in some embodiments, the raw or derived parameters are communicated (e.g., via communications module 52B in conjunction with communications interface 96) to other devices that are used to determine health and/or well-being of one or more of the occupants of the vehicle 10, including to the wearable device 36 (FIG. 2), mobile device 60 (FIG. 3), or one or more devices of the cloud(s) 18, 26 (FIG. 1). The communications functionality of the communications module 52B generally enables communications among devices connected to one or more networks (NW) (e.g., personal area network, local wireless area network, wide area network, cellular network, etc.), including enabling web-browsing and/or access to cloud services through the use of one or more APIs.

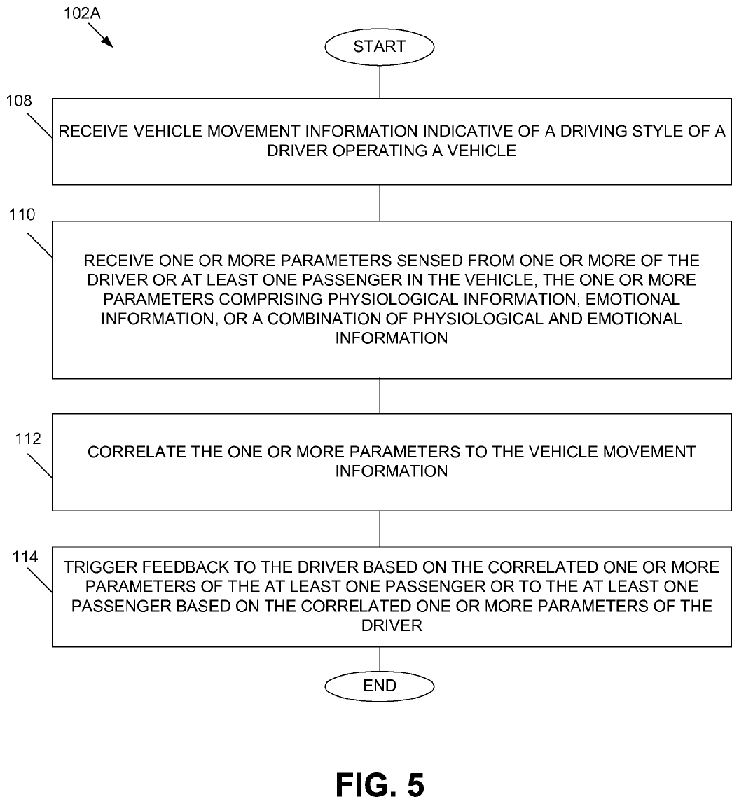

The driving style correlator module 102 comprises executable code (instructions) to receive sensor data (e.g., from sensors 100 and/or from other devices) that senses the health and/or well-being parameters of the driver and/or passenger, sensor data pertaining to vehicle motion information (e.g., from sensors 100 that measure vehicular movement that is reflective of the driving style of the driver), correlates the driving style/vehicle motion to the parameters (e.g., based on a stimulus-response association that is proximal in time and similar in context), and triggers feedback (e.g., causing activation of feedback mechanisms of the user interface 98 and/or communicating signals to trigger other non-vehicular devices that perform the feedback). With continued reference to FIG. 4, and referring also to FIG. 5, shown is an example vehicle occupant interaction method 102A corresponding to functionality of the driving style correlator module 102, which includes receiving vehicle movement information indicative of a driving style of a driver operating a vehicle (108); receiving one or more parameters sensed from one or more of the driver or at least one passenger in the vehicle, the one or more parameters comprising physiological information, emotional information, or a combination of physiological and emotional information (110); correlating the one or more parameters to the vehicle movement information (112); and triggering feedback to the driver based on the correlated one or more parameters of the at least one passenger or to the at least one passenger based on the correlated one or more parameters of the driver (114). The physiological and emotional information can include stress, anxiety, sickness, frustration, anger, etc. that is determined from one or more physiological parameters (e.g., skin conductance, heart rate, heart rate variability, etc.). In one embodiment, the feedback may be presented in a way that is inconspicuous. For instance, the driver may be alerted (e.g., visually, audibly, and/or via tactile stimuli) to anxiety or changes in (e.g., increasing) anxiety of the passenger as correlated by the module 102 to driving style, such feedback presented in a manner that is transparent to the passenger (e.g., a tactile or increasingly rapid or intensified tactile stimuli communicated to the wearable device 36 of the user, or caused by activation of a vibratory motor in the steering wheel), thus influencing a change in the driver's style of driving (e.g., slower speeds, less quick turns, etc.). In some embodiments, the driving style correlator module 102 may receive (e.g., via the communications interface 150 in conjunction with the communications module 52B) one or more parameters from wearable devices 36 of one or more passengers of the vehicle 10 (FIG. 1), and respond accordingly. For instance, one of two passengers may be experiencing motion sickness, and the driving style correlator module 102 correlates that sickness to the driving style, and triggers feedback to the driver. Changes in condition and/or status of the monitored occupant may be reflected with changes in intensity of the feedback and/or changes in the manner of feedback (e.g., buzzer transitions to beeps, or vice versa). In some embodiments, the feedback may include instruction as to the correlation, such as an alert of the motion sickness and a correlation to continual abrupt stops and starts. In some embodiments, the feedback may include a recommendation in lieu of, or in addition to, the correlated feedback (e.g., “driver—passenger B is getting motion sickness—it is recommended that you start and stop gradually to avoid causing the motion sickness”). In some embodiments, parameters may be received from occupants of other vehicles. For instance, another driver A within wireless range of the vehicle processing unit 86 may detect that the driver B is getting angry, and after correlating that anger to the driver's driving style, feedback is triggered that alerts the driver A and/or recommends, to driver A, a driving style that reduces this anger of the driver B of the other vehicle. In some embodiments, the driving style correlator module 102 may access remote databases that include the health and/or well-being of occupants of other vehicles in the geographical location (e.g., via the communication of geographical coordinates and current time), and based on that information, a similar result ensues.

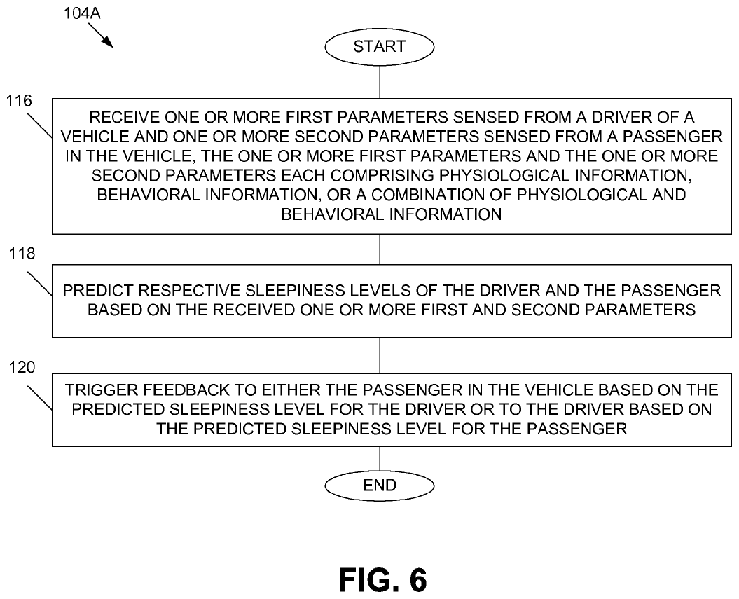

The sleepiness prediction module 104 comprises executable code (instructions) to receive sensor data (e.g., from sensors 100 and/or from other devices of occupants within the vehicle 10 (FIG. 1)) that senses health and/or well-being parameters of the driver and passenger, predicts a level of sleepiness of the occupants, and triggers feedback alerting one of the occupants of the sleepiness state of the other. With continued reference to FIG. 4, and referring also to FIG. 6, shown is an example vehicle occupant interaction method 104A corresponding to functionality of the sleepiness prediction module 104, which includes receiving one or more first parameters sensed from a driver of a vehicle and one or more second parameters sensed from a passenger in the vehicle, the one or more first parameters and the one or more second parameters each comprising physiological information, behavioral information, or a combination of physiological and behavioral information (116); predicting respective sleepiness levels of the driver and the passenger based on the received one or more first and second parameters (118); and triggering feedback to either the passenger in the vehicle based on the predicted sleepiness level for the driver or to the driver based on the predicted sleepiness level for the passenger (120). For instance, the prediction performed by the sleepiness prediction module 104 may be based on a comparison of the parameters indicating sleepiness (e.g., based on sensed breathing rate, such as visually detected expansion in the chest cavity and/or abdomen alone or in combination with other parameters, including heart rate data, sleep history (e.g., as determined from the wearable device 36), driver history (e.g., elapsed time the driver has been driving), time of day, etc.) with a threshold level of sleepiness. The threshold level of sleepiness may be based on learned behavior (e.g., via monitoring and recording by the wearable device 36 (FIG. 2) and/or recording to the clouds 18, 26 (FIG. 1)) providing sleep habits, including normal sleep behavior of the user, and/or based on knowledge of population-based statics for like-individual characteristics (e.g., age, gender, occupation, sleep statistics for those demographics, hour of the day, etc.), such as accessed from remote databases. For instance, in the case where the driver wishes to have the passenger remain awake, based on the sleepiness prediction levels exceeding the sleepiness threshold, it is determined that the passenger is sleepy or has even fallen asleep, which prompts feedback to the driver, the feedback permitting the driver to awaken the passenger. Similarly, based on the predicted levels for the driver exceeding the sleepiness threshold, feedback is presented to the passenger to help keep the driver awake (e.g., prompting the passenger to turn up the radio, or chat more with the driver). Although a single threshold is described, in some embodiments, multiple thresholds may be used, wherein the exceeding of a sleepiness level of one threshold versus another threshold triggers a different type of feedback (e.g., quiet or dim light as feedback for a first threshold is replaced with a loud or higher-intensity light for a second feedback).

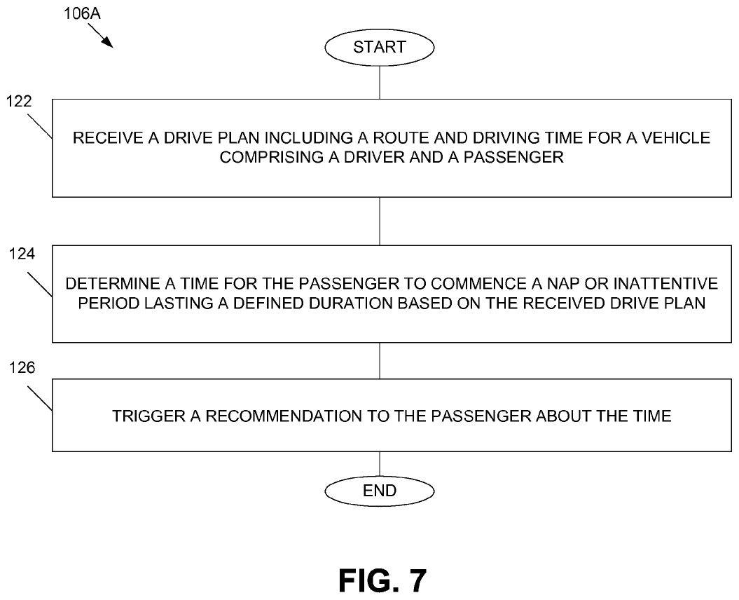

The nap/alertness (NA) module 106 comprises executable code (instructions) to receive a drive plan and recommend a time for a passenger to either take a nap or at least permit inattentiveness during a drive. With continued reference to FIG. 4, and referring also to FIG. 7, shown is an example vehicle occupant interaction method 106A corresponding to functionality of the nap/alertness module 106, which includes receiving a drive plan including a route and driving time for a vehicle comprising a driver and a passenger (122), wherein the route and driving time may include data related to the route and/or driving time such as current traffic data, predicted traffic data, current weather data, predicted weather data, current construction or road hazard data, future construction or road hazard data, and/or the like; determining a time for the passenger to commence a nap or inattentive period lasting a defined duration based on the received drive plan (124); and triggering a recommendation to the passenger about the time (126). The nap/alertness module 106 determines the time based on one or any combination of data regarding the route and driving time, the passenger(s), and/or the driving, such as information about a sleep behavior of the driver and/or passenger, travel safety and/or complexity along a given route, elapsed driving time for the driver, time of day (e.g., evening, morning, afternoon), traffic conditions, the presence of construction/lane closures, and/or weather. In some embodiments, the nap/alertness module 106 may access one or more of such information from a remote database. In some embodiments, weather information may be accessed from sensors and/or devices within or on the exterior of the vehicle 10 (FIG. 1). The plan comprises in one embodiment a planned route, the planned route comprising at least a beginning point and a destination and a path between the two points. The plan may be loaded into the vehicle logic via verbal or text-inputted commands, or transferred from a map app in some embodiments (or downloaded from the cloud(s) 18, 26). The planned route and driving time(s) are considered in scheduling when the passenger can best take a nap (e.g., to be fresh and alert when the passenger switches roles with the driver). As indicated above, the schedule recommends naps or at least allows for the passenger to be inattentive) on safer stretches of the route. Safe stretches may include stretches where there is a lower accident incidence and/or are less challenging for a driver (e.g., as determined from access to a remote database access). One goal in the recommendation is safe travel.

Note that the methods 102A, 104A, and 106A may be implemented according to corresponding modules 102, 104, and 106, respectively, as executed by one or more processors. In one embodiment, the methods 102A, 104A, and/or 106A may be implemented on a non-transitory computer readable medium that is executed by one or more processors (e.g., in the same device or distributed among plural devices). Similarly, in some embodiments, the methods 102A, 104A, and/or 106A may be implemented within a single device (e.g., located within the vehicle 10 (FIG. 1) or located remote from the vehicle 10), or implemented by plural devices located within and/or external to the vehicle 10.

Referring back again to FIG. 4, execution of the application software 46B may be implemented by the processor 88 under the management and/or control of the operating system (or in some embodiments, without the use of the OS). The processor 88 (or processors) may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the vehicle processing unit 86.

When certain embodiments of the vehicle processing unit 86 are implemented at least in part with software (including firmware), as depicted in FIG. 4, it should be noted that the software can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the vehicle processing unit 86 are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

Any process descriptions or blocks in flow diagrams should be understood as representing modules, segments, or portions of code which include one or more executable instructions for implementing specific logical functions or steps in the process, and alternate implementations are included within the scope of the embodiments in which functions may be executed out of order from that shown or discussed, including substantially concurrently or in reverse order, depending on the functionality involved, as would be understood by those reasonably skilled in the art of the present disclosure.

In an embodiment, a claim to a first apparatus comprising: a memory comprising instructions; and one or more processors configured to execute the instructions to: receive vehicle movement information indicative of a driving style of a driver operating a vehicle; receive one or more parameters sensed from one or more of the driver or at least one passenger in the vehicle, the one or more parameters comprising physiological information, emotional information, or a combination of physiological and emotional information; correlate the one or more parameters to the vehicle movement information; and trigger feedback to the driver based on the correlated one or more parameters of the at least one passenger or to the at least one passenger based on the correlated one or more parameters of the driver.

In an embodiment, the first apparatus according to the preceding claim, wherein the parameters correspond to one or any combination of heart rate, heart rate variability, electrodermal activity, accelerometer data, indicators of stress, indicators of anxiety, or indicators of motion sickness.

In an embodiment, the first apparatus according to any one of the preceding claims, wherein the one or more processors are configured to execute the instructions to trigger the feedback by communicating a signal to one or any combination of a tactile device, visual device, audio device, or audio-visual device, wherein the feedback comprises one or any combination of tactile feedback, visual feedback, or audio feedback.

In an embodiment, the first apparatus according to any one of the preceding claims, wherein communicating the signal comprises communicating the signal without alerting the passenger of the feedback to the driver or without alerting the driver to the feedback to the at least one passenger.

In an embodiment, the first apparatus according to any one of the preceding claims, wherein the feedback to the driver is configured to influence a change in the driving style and the feedback to the at least one passenger is configured to influence a change in behavior of the at least one passenger.

In an embodiment, the first apparatus according to any one of the preceding claims, wherein the one or more processors are further configured to execute the instructions to: receive one or more parameters sensed from one or more additional passengers in the vehicle, the one or more parameters comprising physiological information, emotional information, or a combination of physiological and emotional information, wherein the correlation and the trigger is based on the one or more additional passengers.

In an embodiment, the first apparatus according to any one of the preceding claims, wherein the one or more processors are further configured to execute the instructions to: receive one or more parameters sensed from one or more occupants in one or more other vehicles, the one or more parameters comprising physiological information, emotional information, or a combination of physiological and emotional information, wherein the correlation and the trigger is based on the one or more occupants.

In an embodiment, the first apparatus according to any one of the preceding claims, wherein the one or more processors are further configured to execute the instructions to: receive additional vehicle movement information indicative of an adjusted driving style of the driver operating the vehicle subsequent and proximal in time to the trigger.

In an embodiment, an apparatus claim according to any one of the preceding claims, wherein the one or more processors and the memory are located within a structure of the vehicle, in a mobile device within the vehicle, in a wearable device within the vehicle, or in a device external to the vehicle.

In an embodiment, a method implementing functionality of any one of the preceding first apparatus claims.

In an embodiment, a non-transitory computer readable medium that, when executed by one or more processors, causes implementation of the functionality of any one of the preceding first apparatus claims.

In an embodiment, a claim to a second apparatus comprising: a memory comprising instructions; and one or more processors configured to execute the instructions to: receive one or more first parameters sensed from a driver of a vehicle and one or more second parameters sensed from a passenger in the vehicle, the one or more first parameters and the one or more second parameters each comprising physiological information, behavioral information, or a combination of physiological and behavioral information; predict respective sleepiness levels of the driver and the passenger based on the received one or more first and second parameters; and trigger feedback to either the passenger in the vehicle based on the predicted sleepiness level for the driver or to the driver based on the predicted sleepiness level for the passenger.

In an embodiment, the second apparatus according to the preceding claim, wherein the one or more processors are further configured to execute the instructions to: compare the respective predicted sleepiness levels to a corresponding sleepiness threshold, wherein the trigger is further based on the comparison.

In an embodiment, the second apparatus according to any one of the preceding second apparatus claims, wherein the feedback to the driver is configured to alert the driver that the passenger has exceeded a sleepiness threshold or has fallen asleep.

In an embodiment, the second apparatus according to any one of the preceding second apparatus claims, wherein the feedback to the passenger is configured to alert the passenger that the driver has exceeded a sleepiness threshold.

In an embodiment, the second apparatus according to any one of the preceding second apparatus claims, wherein the one or more processors are configured to execute the instructions to trigger the respective feedback by communicating a signal to one or any combination of a tactile device, visual device, audio device, or audio-visual device, wherein the feedback comprises one or any combination of tactile feedback, visual feedback, or audio feedback.

In an embodiment, the second apparatus according to any one of the preceding second apparatus claims, wherein the one or more processors and the memory are located within a structure of the vehicle, in a mobile device within the vehicle, in a wearable device within the vehicle, or external to the vehicle.

In an embodiment, a method implementing functionality of any one of the preceding second apparatus claims.

In an embodiment, a non-transitory computer readable medium that, when executed by one or more processors, causes implementation of the functionality of any one of the preceding second apparatus claims.

In an embodiment, a claim to a third apparatus, comprising: a memory comprising instructions; and one or more processors configured to execute the instructions to: receive a drive plan including a route and driving time for a vehicle comprising a driver and a passenger; determine a time for the passenger to commence a nap or inattentive period lasting a defined duration based on the received drive plan; and trigger a recommendation to the passenger about the time.

In an embodiment, the third apparatus according to the preceding third apparatus claim, wherein the one or more processors are further configured to execute the instructions to determine the time based on one or any combination of information about a sleep behavior of the driver, information about a sleep behavior of the passenger, information about the safety of travel along the route, information about complexity of travel along the route, elapsed driving time by the driver, time of day, traffic, construction, or weather.

In an embodiment, the third apparatus according to any one of the preceding third apparatus claims, wherein at least one of the information is received from a source external to the vehicle.

In an embodiment, the third apparatus according to any one of the preceding third apparatus claims, wherein the one or more processors are further configured to execute the instructions to trigger feedback to help the passenger stay attentive outside of the nap duration, wherein the one or more processors are configured to execute the instructions to trigger the feedback by communicating a signal to one or any combination of a tactile device, visual device, audio device, or audio-visual device, wherein the feedback comprises one or any combination of tactile feedback, visual feedback, or audio feedback.

In an embodiment, the third apparatus according to any one of the preceding third apparatus claims, wherein the one or more processors and the memory are located within the vehicle or external to the vehicle.

In an embodiment, a method implementing functionality of any one of the preceding third apparatus claims.

In an embodiment, a non-transitory computer readable medium that, when executed by one or more processors, causes implementation of the functionality of any one of the preceding third apparatus claims.

Note that in the embodiments described above, two or more embodiments may be combined. For instance, a single apparatus may combine functionality of the first, second, and/or third apparatus.

Note that various combinations of the disclosed embodiments may be used, and hence reference to an embodiment or one embodiment is not meant to exclude features from that embodiment from use with features from other embodiments.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

At least the following is claimed:

1. An apparatus, comprising:
a memory comprising instructions; and
one or more processors configured to execute the instructions to:
receive vehicle movement information indicative of a driving style of a driver operating a vehicle;
receive one or more parameters sensed from one or more of the driver or at least one passenger in the vehicle, the one or more parameters comprising physiological information, emotional information, or a combination of physiological and emotional information;
correlate the one or more parameters to the vehicle movement information; and
trigger feedback to the driver based on the correlated one or more parameters of the at least one passenger or to the at least one passenger based on the correlated one or more parameters of the driver.

2. The apparatus of claim 1, wherein the parameters correspond to one or any combination of heart rate, heart rate variability, electrodermal activity, accelerometer data, indicators of stress, indicators of anxiety, or indicators of motion sickness.

3. The apparatus of claim 1, wherein the one or more processors are configured to execute the instructions to trigger the feedback by communicating a signal to one or any combination of a tactile device, visual device, audio device, or audio-visual device, wherein the feedback comprises one or any combination of tactile feedback, visual feedback, or audio feedback.

4. The apparatus of claim 3, wherein communicating the signal comprises communicating the signal without alerting the passenger of the feedback to the driver or without alerting the driver to the feedback to the at least one passenger.

5. The apparatus of claim 3, wherein the feedback to the driver is configured to influence a change in the driving style and the feedback to the at least one passenger is configured to influence a change in behavior of the at least one passenger.

6. The apparatus of claim 1, wherein the one or more processors are further configured to execute the instructions to:
receive one or more parameters sensed from one or more additional passengers in the vehicle, the one or more parameters comprising physiological information, emotional information, or a combination of physiological and emotional information, wherein the correlation and the trigger is based on the one or more additional passengers.

7. The apparatus of claim 1, wherein the one or more processors are further configured to execute the instructions to:
receive one or more parameters sensed from one or more occupants in one or more other vehicles, the one or more parameters comprising physiological information, emotional information, or a combination of physiological and emotional information, wherein the correlation and the trigger is based on the one or more occupants.

8. The apparatus of claim 1, wherein the one or more processors are further configured to execute the instructions to:
receive additional vehicle movement information indicative of an adjusted driving style of the driver operating the vehicle subsequent and proximal in time to the trigger.

9. The apparatus of claim 1, wherein the one or more processors and the memory are located within a structure of the vehicle, in a mobile device within the vehicle, in a wearable device within the vehicle, or in a device external to the vehicle.

10. An apparatus, comprising:
a memory comprising instructions; and
one or more processors configured to execute the instructions to:
receive one or more first parameters sensed from a driver of a vehicle and one or more second parameters sensed from a passenger in the vehicle, the one or more first parameters and the one or more second parameters each comprising physiological information, behavioral information, or a combination of physiological and behavioral information;
predict respective sleepiness levels of the driver and the passenger based on the received one or more first and second parameters; and
trigger feedback to either the passenger in the vehicle based on the predicted sleepiness level for the driver or to the driver based on the predicted sleepiness level for the passenger.

11. The apparatus of claim 10, wherein the one or more processors are further configured to execute the instructions to:
compare the respective predicted sleepiness levels to a corresponding sleepiness threshold, wherein the trigger is further based on the comparison.

12. The apparatus of claim 10, wherein the feedback to the driver is configured to alert the driver that the passenger has exceeded a sleepiness threshold or has fallen asleep.

13. The apparatus of claim 10, wherein the feedback to the passenger is configured to alert the passenger that the driver has exceeded a sleepiness threshold.

14. The apparatus of claim 10, wherein the one or more processors are configured to execute the instructions to trigger the respective feedback by communicating a signal to one or any combination of a tactile device, visual device, audio device, or audio-visual device, wherein the feedback comprises one or any combination of tactile feedback, visual feedback, or audio feedback.

15. The apparatus of claim 10, wherein the one or more processors and the memory are located within a structure of the vehicle, in a mobile device within the vehicle, in a wearable device within the vehicle, or external to the vehicle.

16. An apparatus, comprising:
a memory comprising instructions; and
one or more processors configured to execute the instructions to:
receive a drive plan including a route and driving time for a vehicle comprising a driver and a passenger;
determine a time for the passenger to commence a nap or inattentive period lasting a defined duration based on the received drive plan; and
trigger a recommendation to the passenger about the time.

17. The apparatus of claim 16, wherein the one or more processors are further configured to execute the instructions to determine the time based on one or any combination of information about a sleep behavior of the driver, information about a sleep behavior of the passenger, information about the safety of travel along the route, information about complexity of travel along the route, elapsed driving time by the driver, time of day, traffic, construction, or weather.

18. The apparatus of claim 17, wherein at least one of the information is received from a source external to the vehicle.

19. The apparatus of claim 16, wherein the one or more processors are further configured to execute the instructions to trigger feedback to help the passenger stay attentive outside of the nap duration, wherein the one or more processors are configured to execute the instructions to trigger the feedback by communicating a signal to one or any combination of a tactile device, visual device, audio device, or audio-visual device, wherein the feedback comprises one or any combination of tactile feedback, visual feedback, or audio feedback.

20. The apparatus of claim 16, wherein the one or more processors and the memory are located within the vehicle or external to the vehicle.
